(19) United States
(12) Patent Application Publication
(10) Pub. No.: US 2002/0099037 A1
(43) Pub. Date: Jul. 25, 2002

(54) Method for alleviating syndromes and conditions of discomfort of the mammalian intestinal and genito-urinary tracts

(75) Inventors: Alan E. Kligerman, Egg Harbor Township, NJ (US); Sarah Finnegan, Mays Landing, NJ (US)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: AkPharma, Inc., Pleasantville, NJ

(21) Appl. No.: 10/051,853

(22) Filed: Jan. 17, 2002

Related U.S. Application Data
(60) Provisional application No. 60/262,759, filed on Jan. 19, 2001.

(51) Int. Cl. A61K 31/66

(52) U.S. Cl. 514/129

(57) ABSTRACT

Glycerophosphates, particularly calcium glycerophosphate (CGP), have been found to mitigate certain abdominal-area physical problems including irritable bowel syndrome and urinary urgency. It is believed that calcium and glycerophosphate, taken orally, are introduced into the human and other animal system such that the glycerophosphate and/or calcium exert a damping, soothing, irritant-interdictive or antispasmodic action on the intestinal, urinary bladder and other smooth muscle organs. Using glycerophosphates, a method is provided for alleviating, palliating, and reducing the syndromes and conditions of discomfort resulting from a variety of diseases, including irritable bowel syndrome, interstitial cystitis, inflammatory bowel disease, fibromyalgia, urinary urgency, benign prostatic hypertrophy, vulvodynia and external genital pain. It is also suggested that muscular energy supplied via glycolysis, the source of anaerobic energy, may be facilitated by the administration of a glycerophosphate moiety to the body's glycerophosphate shuttle.

### CROSS-REFERENCE TO RELATED APPLICATIONS

This application claims the benefit of U.S. Provisional Application No. 60/262,759, filed Jan. 19, 2001.

### BACKGROUND OF THE INVENTION

Inflammatory Bowel Syndrome (IBS) is an ailment of the intestines which is characterized by high motility of the small and large intestines to a degree that may be characterized as ‘spasms’. These muscular spasms do not always move smoothly, in concert, or even in the same direction; sometimes the peristaltic motions are adverse to one another, causing intestinal bulging in the area between them. There is almost always pain and cramping involved, which may be accompanied by diarrhea or constipation. These effects have been likened to those associated with severe lactose intolerance (Merck Manual of Diagnosis and Therapy On-Line, “Differential Diagnosis,” p.5, Sep. 29, 2000). The spasms are not the same as the normal non-peristaltic movements of colonic smooth muscle, called halustrations, which appear to have the purpose of maximizing contact of the colonic contents with the lining of the colon walls, thus promoting absorption of nutrients. Rather, sufferers of IBS have the perception of pain and cramping as well as constipation and/or diarrhea. There have also been reports that acidic foods make the symptoms of IBS worse or may even “trigger” this ailment.

IBS has no apparent etiology; that is, it does not readily disclose any particular physical clue as to what causes it. The Merck Manual (Sep. 30, 2000) explains that the cause of irritable bowel syndrome is unknown and that no anatomic cause has been found. The unknown cause has also been expressed in the online publication of the U.S. Government's NIDDK National Digestive Diseases Information Clearinghouse. Furthermore, the publication, Best and Taylor's Physiological Basis of Medical Practice, 12^{th} Edition, explains that IBS is probably not a single nosological entity and thus is difficult to describe in physiological or anatomical terms.

Potential causes for IBS have been offered, including disorders of anxiety, panic, depression and somatization, as well as diet, drugs, hormones and caloric density of food (Merck Manual). Further, IBS has been defined as a “. . . clinically and physiologically undefined syndrome characterized by symptoms thought to represent abnormalities in colonic motor function . . . The symptoms may reflect emotional disturbances . . . ” (Best and Taylor, pp. 642-643). There is still no insight regarding a specific cause, cure or means of palliation of this problem.

Interstitial cystitis (IC) is a urinary bladder disease for which it has been reported that the drinking of wine, coffee, fruit juices, or other acidic drinks causes ‘flares’ in the urinary bladder of interstitial cystitis (IC) sufferers. As discussed in further detail below, the use of calcium glycerophosphate with IC patients has been established as being of important pain-preventive help to them. IC has no known cause and no known etiological cure; etiological markers are scarce.

Calcium glycerophosphate (CGP) is also known as 1,2,3-propanetriol, mono(dihydrogen phosphate) calcium salt (1:1), calcium glycerinophosphate, calcium phosphoglycerate and Neurosin®. It has a molecular formula of C₃H₇CaO₆P and a formula weight of 210.14 (anhydrous). It may exist as a hydrate, including the monohydrate and the dihydrate. Three CGP isomers exist, namely β-glycerophosphoric acid calcium salt ((HOCH₂)₂CHOPO₃Ca) and D(+) and L(−)-α-glycerophosphoric acid calcium salt (HOCH₂CH(OH)CH₂OPO₃Ca). Any one isomer, or any combination of two or more isomers, may be used as the CGP according to this invention. A commercially available form of CGP is a mixture of calcium β- and DL-α-glycerophosphates, and this is a preferred form of CGP according to the invention. The preferred form of CGP is food grade CGP according to Foods Chemical Codex (FCC) III, and may be obtained from Gallard Schlesinger Company, Carl Place, N.Y. 11514, which is a distributor for the Dr. Paul Lohmann GmbH KG of Emmerthal, Germany. CGP is also available from Astha Laboratories Pvt, Ltd, Hyderabad—500 018, India and Seppic Inc., 30 Two Bridges Road, Fairfield, N.J.

It is known that calcium glycerophosphate (CGP) has a hydrogen-ion binding capability, the means by which, in vivo and in vitro, CGP neutralizes the acid in foods and beverages and, topically applied, acidic conditions on the skin. The use of CGP to neutralize the acid in foods and beverages has been described in U.S. Pat. Nos. 5,665,415 and 5,869,119 of Kligerman, et al., which are herein incorporated by reference in their entirety. The use of CGP in neutralizing the acid of skin is described in U.S. Pat. No. 5,972,321 of Kligerman, et al., which is also incorporated herein by reference.

It is known that calcium plays a major role in neurotransmission and it has been hypothesized that the oral consumption of calcium will assist in conditions relating to malfunctioning or impaired neural transmission through an astrocyte common to several synapses (A Theory of Cortical Neuron-Astrocyte Interaction, D. S. Antanitus, Harvard Medical School. pp. 1-11, 1998).

In addition to the calcium cation, the glycerophosphate (GP) organic anion is also known to take part in crucial processes of human physiology. Specifically, glycerophosphates may assist in transmembrane transport of neutralized, non-damaging H⁺ ions into harmless and productive pathways such as glycolysis, the process which produces anaerobic energy for muscle contraction as well as for neurotransmission of astrocytes. (Antanitus, 1998; “Potassium Glycerophosphate Looking For a Superb Energy Enhancer?” Nutrinews GCI Nutrients, pg 4 .online. Jul. 10, 2000; “Neuro Intensive Care; C-HNordstrom et al., Karolinska Institute, Sweden; Microdialysis in Intensive Care online Oct. 27, 2000). Potassium is also recognized as an irritating cation when free in the urinary bladder, and possibly in other locations in the body (“Diagnosing Interstitial Cystitis in Women”; D. L. Myers, et al.; Women's Health in Primary Care, Vol. 1, No. 2, pg. 135, Apr. 2001).

Glycerophosphate is a metabolic intermediate in the conversion of foods to biologically useful form and it is manufactured de novo in the body of humans and presumably other mammals. However, the glycerophosphate in a body is not presented to the same physical locales as the orally ingested glycerophosphate, at least initially. The value of GP has been recognized by experts in the field as being safe for consumption, even in large amounts, and for providing a favorable effect on the central nervous system and on metabolic processes in the body (Fedorov, Y. A.; “The Effect of Phosphorus-calcium and Fluorine Compounds on Experimental Dental Caries in White Rats”; Doklady Academii Nauk SSSR; 137, #6, 1481-1484, 1961).

In one research study from 1951, it was shown that sodium and magnesium glycerophosphates behaved as uterine anti-spasmodic agents. It was hypothesized that the glycerophosphate radical was responsible for the anti-spasmodic action on the smooth muscle. Such anti-spasmodic behavior was found, however, to exclude the intestine (“Experimental Pharmacological Study of the Anti-Spasmodic Action of Sodium Beta-Glycerophosphate on the uterus;” D. U. Aragon; Anales fac. farm y bioquim., Univ. nacl. Mayor San Marcos 2:417-429 (1951)). Such experimentation was restricted to in vitro work on excised animal uterus tissue and not on live mammals.

The favorable effects of CGP on the nervous system, as well as significant survival effects of CGP and other GP compounds on neurons when compared with other substances such as calcium chloride and ‘normal’ non-reagent added media, have been discussed in the literature. Further, beneficial effects of CGP in playing a fundamental role in the generation of cell membranes and vital organ tissue assembly have been proposed: CGP has also been demonstrated in vitro to enhance the proliferation and differentiation of astrocytes (J. Boucraut and R. Steinschneider; “Evaluation de l'influence du glycerophosphate de calcium sure les cellules corticales (astrocytes et neurones) en culture”; Laboratoire d'immunopathologie-Universite d'Aix-Marseilles; 1995; J. Boucraut and R. Steinschneider; “Effet du glycerophosphate de calcium du glycerophosphate de magnesium, de l'acide glycerophosphorique et du calcium sur la survie et la differenciation des neurones de rat en culture”; Laboratoire d'immunopathologie- Universite d'Aix-Marseilles; Givocal® Nutritional; Ingredient, Carrier of Highly Bioavailable Calcium and Phosphorus; Seppic Inc.; 9/97).

Calcium glycerophosphate has been used as an electrolyte in a nutritional product known as “Alpha ENF” (available from Nutramed at http://www.nutramed.com), which is claimed to be effective for treating ailments such as fibromyalgia and irritable bowel syndrome. However, the product, which also contains sixteen amino acids, fourteen vitamins and choline bitartrate, is merely a food replacement and, according to the manufacturers, is designed only to bring about immune responses to food and other antigens.

As a result of the reports that CGP has been helpful for suffers of IC, two calcium glycerophosphate studies and one less formal survey were performed to investigate the effect of CGP on the symptoms of IC. The first study is described in, Bologna, et al, “Survey of the Effect of PRELIEF® [CGP] on Food-Related Exacerbation of Interstitial Cystitis Symptoms”(1998-99). The results of this study were that for a number of key exacerbant foods and beverages, 70.4% of the patients reported a reduction in pain and discomfort, while 61.3% reported a reduction in [xurinary] urgency.

The second study is analyzed in Tu, Polansky, et al. “A Retrospective Analysis of Calcium Glycerophosphate (Prelief® in the Treatment of Food-Sensitive Interstitial Cystitis Patients”; (Quebec Urological Association, 2001). Patients in the study found that as a result of consuming CGP, pain decreased from 76.7% to 41% and urinary urgency decreased from 79.5% to 51%.

Finally, the survey is described in P. Jones, “The Therapeutic Effect of Calcium Glycerophosphate (Prelief® in Interstitial Cystitis, A Survey of Interstitial Cystitis Support Group (ICSG-LJK) Members”(January, 2000). Briefly, 69% of the participants found Prelief® (CGP) to be beneficial and 81% found they were enabled to tolerate a wider variety of foods of an acidic nature.

Traditional treatments for IBS and related ailments are often drugs which are designed to treat the symptoms, with little apparent consideration or regard as to whether the symptoms might in fact be necessary defensive responses by the organ(s) involved. In other words, drugs are often targeted against the sites of specific organ pain. However, the pain may not be the result of any etiological problem with the particular organ, but may instead be a result of brain interpretation confusion when two or more organs share a common neural pathway. A recently developed drug to treat IBS, Lotronex® (Glaxo SmithKline), was designed to target the intestinal musculature. Unfortunately, in some cases, uses of Lotronex® led to intestinal ischemic collapse, triggering warnings about the drug's use. In December, 2000, as a result of further reports of ischemic colonic collapse in women and of some deaths, this product was withdrawn from the market.

Therefore, there remains a need in the art for a method of alleviating or interdicting the pain and symptoms of patients suffering from IBS, which to some degree may affect up to 10-12% of the general population, or up to an estimated 35 million persons in the United States alone. Additionally, there is a need for a method of preventing or palliating the pain and symptoms of syndromes or conditions which may be related to IBS by common neural pathways, including inflammatory bowel diseases, interstitial cystitis, fibromyalgia, urinary urgency, benign prostatic hypertrophy, vulvodynia and external genital pain

### BRIEF SUMMARY OF THE INVENTION

This invention is directed to a method for interdicting, preventing, palliating, or alleviating a syndrome or a condition of discomfort of a mammalian intestinal or genito-urinary tract resulting from the mammal's consumption of a drug, a food or a beverage. The method involves administering to the mammal a glycerophosphate moiety attached to an ingestible, dissociable ion in an amount effective to reduce the discomfort to a level which allows the mammal to consume the drug, food, or beverage without substantial discomfort. When the syndrome or condition is interstitial cystitis, the glycerophosphate moiety is not calcium glycerophosphate.

The invention also provides a method for interdicting, preventing, palliating, or alleviating causes of pain and symptoms from a disorder of a mammalian intestinal or genito-urinary tract resulting from the mammal's consumption of a drug, a food or a beverage which comprises administering to the mammal a glycerophosphate moiety attached to an ingestible, dissociable ion in an amount effective to impede the causes of pain or symptoms to a level which allows the mammal to consume the drug, food, or beverage without substantial pain or symptoms. When the syndrome or condition is interstitial cystitis, the glycerophosphate moiety is not calcium glycerophosphate.

This invention is also directed to a method for facilitating a smooth muscular operation in a muscle of a mammalian intestinal or genito-urinary tract comprising administering to the mammal an effective amount of a soluble calcium ion attached to an anion in an amount effective to facilitate proper and effective operation of the smooth muscles to a greater or more optimum degree than that which would have occurred in the absence of the calcium ion.

A method is also provided for reducing pain in a mammal suffering from an intestinal or genito-urinary tract disorder which comprises administering to the mammal a glycerophosphate moiety attached to an ingestible, dissociable ion in an amount effective to reduce the pain to a level below that which would have been experienced in the absence of the glycerophosphate moiety.

Additionally, this invention is directed to a method for neutralizing acid in an organ or a part of a gastrointestinal or genito-urinary tract of a mammal below the pyloric valve which comprises administering to the mammal calcium glycerophosphate in an amount effective to increase the pH of the organ or part of the gastrointestinal tract to a level greater than it would have been in the absence of the calcium glycerophosphate.

Moreover, this invention involves a method for relieving acid-sensitive internal epithelial skin or organ irritation in a mammal comprising administering to the mammal calcium glycerophosphate in an amount effective to interdict, prevent, palliate, or reduce a tendency of a symptom or a pain response resulting from the irritation.

Finally, this invention is directed to a method for repairing, assisting, or supporting an anaerobic energy transfer mechanism in a mammal comprising administering to the mammal a glycerophosphate moiety attached to an ingestible, dissociable ion in an amount effective to improve or enhance the energy transfer mechanism of the mammal.

### DETAILED DESCRIPTION OF THE INVENTION

This invention is directed to a method for using glycerophosphates, in particular, calcium glycerophosphate (CGP), to interdict, palliate, prevent or relieve the symptoms of diseases such as irritable bowel syndrome (IBS) and other diseases or conditions of the intestinal and genito-urinary tract. As explained previously, symptoms of IBS or other related syndromes are often exacerbated by the consumption of particular drugs, foods or beverages, and the method according to this invention is directed to addressing this problem. Additionally, this invention is directed to a method for using glycerophosphates for relieving symptoms of these conditions or syndromes which occur in the absence of a food, beverage or drug.

This invention thus involves a method for alleviating a syndrome or a condition of discomfort or for interdicting causes or pain and symptoms from a disorder of a mammalian intestinal or genito-urinary tract both resulting from the mammal's consumption of a drug, a food or a beverage, as well as relief from the same symptoms when the problem has no apparent direct cause or trigger. The method involves administering to the mammal a glycerophosphate moiety attached to an ingestible, dissociable ion in an amount effective to reduce the discomfort or pain to a level which allows the mammal to consume a drug, food, or beverage without substantial discomfort or pain, or to prevent or reduce such pain and/or symptoms regardless of food, beverage or drug consumption. The condition or disorder may be, for example, irritable bowel syndrome, interstitial cystitis, inflammatory bowel disease (such as colitis, diverticulitis, diverticulosis or Crohn's disease), fibromyalgia, urinary urgency, benign prostatic hypertrophy, vulvodynia or external genital pain.

In a preferred embodiment, the ingestible, dissociable ion is calcium and the compound administered to the mammal is CGP. However, any ingestible ion may be used, including, for example, magnesium, potassium and sodium. Preferably, the glycerophosphate moiety is administered orally. It may be administered with foods, beverages or drugs, under which circumstances it may be advantageous to administer an amount proportional to the acidity of such a product, or it may be taken at any time of day or night. It is preferred if the glycerophosphate moiety is administered at intervals during the day, such as at breakfast, lunch, dinner, and upon retiring. If the administration of the glycerophosphate occurs with food intake, it is preferably also ingested with snacks when they are consumed. It may be administered in the form of a tablet, which may be swallowed, or as a granulate, which may be sprinkled on or in foods, beverages, or in a glass of water, for example. No method is preferred, as long as an effective amount of glycerophosphate is administered.

The effective amount of glycerophosphate is preferably between about 0.1 gram and about 3.0 grams, and more preferably between about 0.3 gram and about 1.0 gram per dose. The preferred daily dosage of glycerophosphate is between about 0.6 grams and 18 grams, and more preferably between about 1.8 grams and about 6 grams. However, the number of doses per day and the quantity of glycerophosphate which may be administered to a patient is unlimited. The effective amounts of the glycerophosphate moiety are the same regardless of whether the glycerophosphate is to be administered for alleviating or interdicting pain or symptoms.

This invention also describes a method for facilitating smooth muscular operation in a muscle of a mammalian intestinal or genito-urinary tract which comprises administering to the mammal an effective amount of a soluble calcium moiety attached to an anion in an amount effective to facilitate proper and effective operation of the smooth muscles to a greater or more optimum degree than that which would have occurred in the absence of the calcium moiety.

In a preferred embodiment, the anion is a glycerophosphate radical and the compound administered to the mammal is calcium glycerophosphate. Other possible anions which may be used according to the present invention include carbonate, chloride, citrate, and lactate, for example. It is preferred if the soluble calcium ion is administered orally. The effective amount of calcium is preferably between about 0.1 gram and about 3 grams, and more preferably between about 0.3 gram and about 1.0 gram per usage. Although the calcium ion may be administered as often as desired, it is preferably administered between about once and about eight times per day.

A method is also provided for reducing symptoms and pain in a mammal suffering from an intestinal or genito-urinary tract disorder which comprises administering to the mammal a glycerophosphate moiety attached to an ingestible, dissociable ion in an amount effective to reduce symptoms and the pain to a level below that which would have been experienced in the absence of the glycerophosphate moiety. Such diseases include, but are not limited to irritable bowel syndrome, inflammatory bowel disease (including colitis, diverticulitis, diverticulosis and Crohn's disease), fibromyalgia, urinary urgency, benign prostatic hypertrophy, vulvodynia and external genital pain.

In a preferred embodiment, the ingestible, dissociable ion is calcium and the glycerophosphate moiety is administered orally. Other cations which may be used according to the present invention include sodium, potassium, and magnesium. The effective amount of glycerophosphate is preferably between about 0.1 gram and about 3.0 grams, and more preferably between about 0.3 gram and about 1.0 gram per usage. Although the calcium ion may be administered as often as desired, it is preferably administered between about once and about eight times per day.

This invention also provides a method for neutralizing acid in an organ or a part of a gastrointestinal or genito-urinary tract of a mammal below the pyloric valve which comprises administering to the mammal calcium glycerophosphate in an amount effective to increase a pH of the organ or part of the gastrointestinal tract to a level greater than it would have been in the absence of the calcium glycerophosphate. The organs include, but are not limited to, the intestine and urinary bladder, as well as other organs within the region of the enteric nervous system. It is preferred if the calcium glycerophosphate is administered orally. The calcium glycerophosphate may be administered in the form of tablet or a granulate, including as an excipient accompanying a drug or as a filler in any food product, which may be swallowed or dissolved in water, in food, or in a beverage. There is no preferred method of administration as long as the effective amount of calcium glycerophosphate is administered to the patient.

The effective amount of calcium glycerophosphate is preferably between about 0.1 gram and about 3.0 grams, and more preferably between about 0.3 gram and about 1.0 gram per usage. Although the calcium ion may be administered as often as desired, it is preferably administered between about once and about eight times per day.

A method is also provided for relieving acid-sensitive internal epithelial skin or organ irritation in a mammal comprising administering to the mammal calcium glycerophosphate in an amount effective to reduce a tendency of a symptom or a response to foods, or to relieve, prevent or interdict a physical condition extant regardless of apparent connection to foods when such responses or tendencies produce pain, urinary or bowel urgency or retention (diarrhea or constipation) resulting from the irritation. The response or tendency may be, for example, pain, a muscle spasm, and another symptom, such as diarrhea or constipation. There may also be burning in the urinary bladder or bloating in the intestine.

It is preferred if the calcium glycerophosphate is administered orally and may be administered in the form of a tablet or a granulate, as described above. The effective amount of calcium glycerophosphate is preferably between about 0.1 gram and about 3.0 grams, and more preferably between about 0.3 gram and about 1.0 gram per usage. Although the calcium ion may be administered as often as desired, it is preferably administered between about once and about eight times per day.

The invention will best be described in more detail with respect to the following non-limiting example.

EXAMPLE 1

A ‘pre-study’ IBS survey was taken in a large private gastroenterology practice in southern New Jersey. Each of the patients in the survey consumed a series of 2-tablet doses of CGP, in which each tablet contained 0.335 grams CGP so that each dosage was 0.67 gram of CGP. Two tablets were consumed at breakfast, lunch, and dinner, regardless of the acidity of the foods consumed at the meals, and two additional tablets were consumed with any snack eaten during the day, regardless of the acidity of the snack. The total consumption by each patient varied from 8-10 tablets per day up to 20 tablets per day.

The survey included twelve patients, eight of whom completed graphs of their 28-day experience with CGP. The experiences of the four remaining patients were written up by the medical supervisor based on oral reports. The graphs completed by the patients had a central ‘horizon’ as the base line for any patient on the day before they started the survey, regardless of their pain and/or symptoms. The patients marked daily, in 10% increments and 10% decrements, whether they felt up to 100% better or down to 100% worse as a result of the treatment.

Of the four patients who did not get written up but who verbally reported to the professional medical supervisor, one discontinued the study because there were too many pills. The other reports indicated that the CGP was helpful and effective at relieving symptoms and/or pain. Two of the eight patients who completed graphs were “flatliners”: there was no effect on their pain and/or symptoms as a result of ingesting CGP. One of these patients stopped the treatment after 15 days. Finally, of the remaining six patients, the results were good to remarkable, with some going to 100% improvement and maintaining that level,

Interestingly, three of the patients in the study experienced one day each in which they did not ingest CGP. On those days only, the patients showed a marked condition deterioration, evidenced by the recurrence or increase in pain or symptoms. One of these patients dropped 10-20% on the scale, the second dropped from “50% better” to “50% worse” and the third dropped from “100% better” to “40% worse.” Upon resuming the treatment, all three patients immediately returned to their improved state.

Such results indicate that 75% of the patients showed major improvements in IBS-related symptoms and conditions as a result of treatment with CGP. Additionally, recent anecdotal reports of IBS sufferers who have used CGP corroborate these results.

While not wishing to be bound by theory, these results seem to indicate that glycerophosphate, either preferably in combination with its ionically-bound calcium or with any other ingestible cation, such as magnesium, sodium, or potassium, plays a beneficial role in the body which may be helpful to sufferers of some prominent and wide-spread maladies such as IBS, inflammatory bowel diseases, urinary urgency, fibromyalgia and/or other problems.

It is believed that CGP has a positive effect on patients suffering from, for example, IBS and urinary urgency, which is at least partially attributable to the acid-binding quality of CGP which has been previously discussed. Specifically, the acid receptor sites of the duodenum can sense the fact that acid has ‘breached the defenses of the intestine.’ In other words, the acid-neutralizing capability of the intestines, which exists in the first few inches of the duodenum where the alkaline bile is secreted, appears to have been overwhelmed. The sensors are also possible muscle spasm triggers. The binding of the acid by the CGP prior to its arrival at those sites averts the cause of the problem and therefore prevents the symptoms from occurring.

In addition to the hydrogen ion or acid-binding capability of calcium glycerophosphate, it has been found that there are additional effects of CGP that work independently of, or in conjunction with, the hydrogen ion-binding property. The ability to capitalize on these effects of CGP to treat patients suffering from diseases located in abdominal sections mediated by the pelvic nerve, namely in areas of the intestines, urinary bladder and external genitalia is attractive.

The ionic calcium in CGP is somewhat unique in its solubility when compared with the calcium in antacids, most notably in calcium carbonate. Although in calcium carbonate the calcium cation is also ionically bonded to the carbonate anion, it is only released after a period of time, estimated at 20-30 minutes after ingestion, and only in an acidic milieu. When calcium carbonate is introduced to an acidic stomach, it begins to neutralize the acid and increases the pH of the stomach. Therefore, as the acidity level decreases, the amount of calcium released from the carbonate simultaneously diminishes. When the calcium carbonate further reaches the small intestine, which has a naturally neutral to slightly alkaline environment, the calcium is, again, unavailable for dissolution because of the adversely high pH. Therefore, the possibility or probability exists that calcium from calcium carbonate or from any source whose release is strongly pH-dependent is to at least some degree biologically unavailable.

This is not true about the calcium in calcium glycerophosphate. Specifically, in contrast with calcium carbonate, the calcium in CGP is instantly dissociated from the glycerophosphate (GP) moiety because the calcium is loosely ionically bound. Therefore, it is already available in the mouth, if wetted there, and is additionally available in the stomach, regardless of the pH level in the stomach The calcium ions are rapidly and freely absorbable across the gastric lumen as well as across the small intestinal lumen immediately upon arrival at each site. Since free ionic calcium plays an absolutely essential role in muscular contraction and neural transmission (Best and Taylor, p. 625), it is conjectured that the ‘spike’ of calcium has a beneficial effect on the intestinal spasms of the IBS patient and/or the contractions of the urinary bladder. Therefore, it appears that CGP plays an alternative role to simple acid neutralization, a uniqueness which is further substantiated by the lack of reports in the literature that any calcium carbonate antacid or product is associated with symptomatic relief of diseases such as inflammatory bowel disease and urinary urgency.

One theory is herewith offered which involves both acid neutralization and calcium ion presence. Specifically, the increase of local calcium supply damps down the enteric nervous system response to pressure perceptions of IBS, while CGP is simply better than other antacids at raising the pH of the urine.

It seems probable that by means of neural and/or muscular effect of some kind, the glycerophosphate radical or moiety may exert a beneficial effect on one or more pained organs in and near the abdominal cavity via effects on the musculature and/or nervous system. CGP has been cited in some literature as conferring a beneficial neural effect on the human body and on the central nervous system (Fedorov, Y. A.; The Effect of Phosphorus-calcium and Fluorine Compounds on Experimental Dental Caries in White Rats; Doklady Academii Nauk SSSR; 137 (6), 1481-1484; (1961)). There may be supporting or essential primary physiological roles played by the ionic calcium alone as well as by glycerin alone and/or GP alone or by their metabolites.

Based on these results, it is believed that glycerophosphate, when taken orally and preferably accompanied by the calcium cation, introduces glycerophosphate and calcium into the mammalian system such that the glycerophosphate and/or calcium exert a damping, soothing, irritant-interdictive or anti-spasmodic action on the intestinal, urinary bladder and other smooth muscle organs.

It appears that in some cases at least, the reasons for intestinal spasm have to do with, ironically, a healthy response by the sensitive or particularly vulnerable intestine. It needs, and is acting accordingly, to rid itself of an unwanted presence of some kind of irritating contents (acidic, spicy, etc), because such contents are potentially irritating to the intestinal wall, the normal and desirable milieu within the intestine being pH neutral to slightly alkaline. It is hypothesized that drugs which frustrate or inhibit the intestine's self-protective mechanism are adverse rather than helpful, in the larger picture. In a urinary bladder, the free irritating ion, potassium, may likewise trigger an expulsion (urgency) reaction.

Without wishing to be bound by theory, it further seems probable that in certain patients, there may be malfunctions in the body's anaerobic energy transfer system. These malfunctions, also known as mitochondria myopathies, are diseases associated with enzyme deficiencies. It is believed that a deficiency of the enzyme glycerophosphate dehydrogenase, involved in the glycerophosphate shuttle during glycolysis, is a possible culprit behind these certain ailments. Glycerophosphate dehydrogenase is produced de novo in the body.

This invention thus provides a method for repairing, assisting, or supporting the anaerobic energy transfer system of a mammal which comprises administering to the mammal a glycerophosphate moiety attached to an ingestible, dissociable ion. It is preferred if the dissociable ion is calcium and that the glycerophosphate moiety is administered orally The glycerophosphate moiety is administered in an amount effective to improve or enhance the energy transfer mechanism of the mammal. Preferably, the effective amount of glycerophosphate moiety is between about 0.1 gram and 3.0 grams, and more preferably between about 0.3 gram and 1.0 gram per usage. Although the calcium ion may be administered as often as desired, it is preferably administered between about once and about eight times per day.

It seems probable that the readily available glycerophosphate moiety from ingested calcium glycerophosphate either stimulates, fixes, or bypasses the malfunction, thereby creating a situation in which the glycerophosphate shuttle may proceed without the prior limitation of insufficient glycerophosphate to support the body's anaerobic respiration.

Based on the above-described results and teachings, it appears that oral CGP administration is beneficial for patients with certain ailments, such as IBS and urinary urgency, for example, to interdict, palliate, rectify or prevent occurrence of symptoms from such syndromes Such benefits may be the results of any or all of the components of CGP, including ionic calcium, glycerin, phosphorus, phosphate or glycerophosphate. In some cases, it appears that a single agent may perform more than one function. For example, glycerophosphate may deacidify as well as have an effect on the central nervous system, enteric nervous system, cellular transport and ATP production taking place in all cells. Glycerophosphate may, in addition, sequester various irritants, such as potassium, by binding.

It is possible that the calcium glycerophosphate used for the treatments of the diseases may be acting in several novel and unique ways:

(a) more than one component of CGP may be performing or enhancing a single effect on the body's functioning (such as glycerophosphate and ionic calcium on musculature), or

(b) one single component of CGP may be performing or enhancing more than one effect on the body's functioning (such as glycerophosphate on neural and muscular operation; and on acid- and other ion-binding).

Additionally, for the reasons discussed previously, the use of calcium glycerophosphate in treating many disorders, is attractive because CGP has been shown to be beneficial to the mammalian body and does not bring about side effects which are common with many drugs. Furthermore, the use of CGP to treat such conditions is appealing because the dosage may be controlled depending on the diet and lifestyle of the patient without risk of overdose. In particular, if the CGP is administered to a patient on a dosage schedule which is associated with meals, one who consumes particularly acidic foods may decide to consume a higher dosage of CGP than one who consumes less acidic foods. Such a treatment is also attractive because the beneficial results appear to be quickly recovered even when a dosage is missed, indicating that, over a long-term treatment plan, it is possible that a more moderate dosage will be sufficient to impart the desired results.

This invention thus provides methods for utilizing calcium glycerophosphate or the ionic components thereof to alleviate, palliate and relieve the symptoms of inflammatory bowel syndrome and other diseases which may be related by a common neural pathway. The CGP is used in unique and novel ways, and also is effective in methods for reducing acidity in parts of the mammalian body and for improving smooth muscular functions. This invention thus fulfills a long-felt need in the art for a treatment for patients suffering from a wide-variety of sydromes or conditions which, while not life-threatening, are painful, debilitating and often overwhelming due to impairment or destruction of quality of life. Additionally, highly important from a standpoint of patient safety and with consideration of long-term use in mind, this invention utilizes safe, “GRAS”, non-drug substances, none of which is alien to the mammalian body.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method for interdicting, preventing, palliating, or alleviating a syndrome or a condition of discomfort of a mammalian intestinal or genito-urinary tract resulting from the mammal's consumption of a drug, a food or a beverage comprising administering to the mammal a glycerophosphate moiety attached to an ingestible, dissociable ion in an amount effective to reduce the discomfort to a level which allows the mammal to consume the drug, food, or beverage without substantial discomfort, wherein when the syndrome or condition is interstitial cystitis, the glycerophosphate moiety is not calcium glycerophosphate.

2. The method according to claim 1, wherein the syndrome or condition is selected from the group consisting of irritable bowel syndrome, interstitial cystitis, inflammatory bowel disease, fibromyalgia, urinary urgency, benign prostatic hypertrophy, vulvodynia and external genital pain.

3. The method according to claim 2, wherein the inflammatory bowel disease is selected from the group consisting of colitis, diverticulitis, diverticulosis and Crohn's disease.

4. The method according to claim 1, wherein the ingestible, dissociable ion is calcium.

5. The method according to claim 1, wherein the glycerophosphate moiety is administered orally.

6. The method according to claim 1, wherein the effective amount of glycerophosphate moiety is between about 0.1 gram and about 3.0 grams per dose.

7. A method for interdicting, preventing, palliating, or alleviating causes of pain and symptoms from a disorder of a mammalian intestinal or genito-urinary tract resulting from the mammal's consumption of a drug, a food or a beverage comprising administering to the mammal a glycerophosphate moiety attached to an ingestible, dissociable ion in an amount effective to impede the causes of pain or symptoms to a level which allows the mammal to consume the drug, food, or beverage without substantial pain or symptoms, wherein when the disorder is interstitial cystitis, the glycerophosphate moiety is not calcium glycerophosphate.

8. The method according to claim 7, wherein the disorder is selected from the group consisting of irritable bowel syndrome, interstitial cystitis, inflammatory bowel disease, fibromyalgia, urinary urgency, benign prostatic hypertrophy, vulvodynia and external genital pain.

9. The method according to claim 8, wherein the inflammatory bowel disease is selected from the group consisting of colitis, diverticulitis, diverticulosis and Crohn's disease.

10. The method according to claim 7, wherein the ingestible, dissociable ion is calcium.

11. The method according to claim 7, wherein the glycerophosphate moiety is administered orally.

12. The method according to claim 7, wherein the effective amount of glycerophosphate moiety is between about 0.1 gram and about 3.0 grams per dose.

13. A method for facilitating a smooth muscular operation in a muscle of a mammalian intestinal or genito-urinary tract comprising administering to the mammal an effective amount of a soluble calcium moiety attached to an anion in an amount effective to facilitate a proper and an effective operation of the muscle to a greater or a more optimum degree than that which would have occurred in the absence of the calcium moiety.

14. The method according to claim 13, wherein the muscular operation is a contraction or a relaxation.

15. The method according to claim 13, wherein the anion is a glycerophosphate radical.

16. The method according to claim 13, wherein the calcium moiety is administered orally.

17. The method according to claim 13, wherein the effective amount of calcium moiety is between about 0.1 gram and about 3.0 grams per dose.

18. A method for reducing pain in a mammal suffering from an intestinal or genito-urinary tract disorder comprising administering to the mammal a glycerophosphate moiety attached to an ingestible, dissociable ion in an amount effective to reduce the symptoms and pain to a level below that which would have been experienced in the absence of the glycerophosphate moiety, wherein when the disorder is interstitial cystitis, the glycerophosphate moiety is not calcium glycerophosphate.

19. The method according to claim 18, wherein the disorder is selected from the group consisting of irritable bowel syndrome, inflammatory bowel disease, fibromyalgia, urinary urgency, benign prostatic hypertrophy, vulvodynia and external genital pain.

20. The method according to claim 19, wherein the inflammatory bowel disease is selected from the group consisting of colitis, diverticulitis, diverticulosis and Crohn's disease.

21. The method according to claim 18, wherein the ingestible, dissociable ion is calcium.

22. The method according to claim 18, wherein the glycerophosphate moiety is administered orally.

23. The method according to claim 18, wherein the effective amount of glycerophosphate moiety is between about 0.1 gram and about 3.0 grams per dose.

24. A method for neutralizing acid in an organ or a part of a gastrointestinal or genito-urinary tract of a mammal below a pyloric valve comprising administering to the mammal calcium glycerophosphate in an amount effective to increase a pH of the organ or part of the gastrointestinal tract to a level greater than it would have been in the absence of the calcium glycerophosphate.

25. The method according to claim 24 wherein the organ is selected from the group consisting of an intestine, a urinary bladder, and any organ within an enteric nervous system.

26. The method according to claim 24, wherein the calcium glycerophosphate is administered orally.

27. The method according to claim 24, wherein the effective amount of glycerophosphate moiety is between about 0.1 gram and about 3.0 grams per dose.

28. The method according to claim 24, wherein the calcium glycerophosphate is in a form of a tablet or a granulate.

29. A method for relieving acid-sensitive internal epithelial skin or organ irritation in a mammal comprising administering to the mammal calcium glycerophosphate in an amount effective to interdict, prevent, palliate or reduce a tendency of a symptom or a response resulting from the irritation.

30. The method according to claim 29, wherein the symptom or the response is selected from the group consisting of pain, a muscle spasm, diarrhea, constipation, burning, bloating, and urinary urgency.

31. The method according to claim 29, wherein the effective amount of calcium glycerophosphate is between about 0.1 gram and about 3.0 grams per dose.

32. A method for repairing, assisting, or supporting an anaerobic energy transfer mechanism in a mammal comprising administering to the mammal a glycerophosphate moiety attached to an ingestible, dissociable ion in an amount effective to improve or enhance the energy transfer mechanism of the mammal.

33. The method according to claim 32, wherein the ingestible, dissociable ion is calcium.

34. The method according to claim 32, wherein the glycerophosphate moiety is administered orally.

35. The method according to claim 32, wherein the effective amount of glycerophosphate moiety is between about 0.1 gram and about 3.0 grams per dose.
